(19) United States
(12) Patent Application Publication
(10) Pub. No.: US 2023/0360746 A1
(43) Pub. Date: Nov. 9, 2023

(54) A/B TESTING AND RANDOM CLINICAL TRIALS IN LEARNING MANAGEMENT SYSTEMS

(71) Applicant: Western Governors University, Salt Lake City, UT (US)

(72) Inventors: William Chad Adams, Heber, UT (US); Jason Levin, Sandy, UT (US); Jess Stokes, Holladay, UT (US); Nicholas Clark Johnson, Sandy, UT (US); Gerald Ray Berrett, III, Lehi, UT (US)

(21) Appl. No.: 18/143,755

(22) Filed: May 5, 2023

Related U.S. Application Data
(60) Provisional application No. 63/338,738, filed on May 5, 2022.

(51) Int. Cl. G16H 10/20

(52) U.S. Cl. CPC G16H 10/20

(57) ABSTRACT

A random clinical trial (RCT) management system is disclosed. The system includes a user interface, including a configuration interface configured to receive user input defining parameters for a randomized clinical trial and a dashboard interface configured to output results of the randomized clinical trial. The system includes an analysis engine configured to analyze data from a plurality of educational modules associated with a learning management system (LMS) course, including a control educational module and at least one variation educational module correlated to the control educational module Analyzing is performed based on user input received at the configuration interface. The analysis engine randomly assigns students interacting with the learning management system to one of the educational modules and reports analysis results to the dashboard interface. The system includes a communications interface configured to be coupled to an LMS.

### CROSS-REFERENCE TO RELATED APPLICATIONS

This application claims the benefit of and priority to U.S. Provisional Patent Application Ser. No. 63/338,738 filed on May 5, 2022 and entitled “A/B Testing And Random Clinical Trials In Learning Management Systems,” and which application is expressly incorporated herein by reference in its entirety.

### BACKGROUND

### Background and Relevant Art

On a national scale, educators lack practical tools to help them efficiently and effectively assess and update instructional materials. As a result, students and educators are spending precious time on lessons and assignments that may not achieve essential learning objectives.

In particular, educators may have an inability to systematically test materials in real classroom settings. Every day, educators make numerous instructional decisions that, over time, define the contours of a students' learning experience. The material educators use to present content, the assignments they use to reinforce content, the instructional modifications and accommodations they provide to scaffold and differentiate their instruction matter for any student's learning. Yet educators, instructional coaches, and curriculum and learning teams have few practical tools to help them test whether and which of these learning materials yield the learning outcomes they seek for their students.

Instead, many of the day-to-day decisions about what to teach, when, and how are either set by distant textbook companies or based on educators' instincts and un-vetted recommendations from colleagues or, increasingly, unregulated sites like Teachers-pay-Teachers or Pinterest. Educators, often lacking the time, tools, and training to effectively test and iterate on instructional materials, rarely use evidence of student learning to guide their selection or improvement of instructional materials. As a result, educators end up using instructional materials that do not maximize their or their students' time and efforts and student learning suffers.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

One embodiment illustrated herein includes a random clinical trial management system for implementing randomized clinical trials in educational systems. The system includes a user interface. The user interface includes a configuration interface configured to receive user input defining parameters for a randomized clinical trial. The user interface further includes a dashboard interface configured to output, to a user, results of the randomized clinical trial, in a fashion where the results are correlated by educational modules. This includes at least one correlation between a control educational module and a variation educational module. The random clinical trial management system further comprising an analysis engine. The analysis engine is configured to analyze data from a plurality of educational modules associated with a learning management system course, including a control educational module and at least one variation educational module correlated to the control educational module comprising a variation to be tested with respect to the control educational module. Analyzing is performed based on user input received at the configuration interface. The analysis engine is configured to randomly assign students interacting with the learning management system to one of the educational modules based on user input received at the configuration interface. The analysis engine is configured to report analysis results to the dashboard interface, where the results can be output and presented to a user. The random clinical trial management system further includes a communications interface configured to be coupled to a learning management system. This allows the random clinical trial management system to receive user input at the configuration interface from users of the learning management system; assign students interacting with the learning management system to educational modules; and provide analysis results through the dashboard interface to users at the learning management system.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates a learning management system coupled to a random clinical trial management system;

FIG. 2 illustrates a learning management system and on-line suite system coupled to a random clinical trial management system;

FIG. 3 illustrates. a process flow between a learning management system and a random clinical trial management system; and

FIG. 4 illustrates a method of performing a random clinical trial in a learning management system environment.

### DETAILED DESCRIPTION

Embodiments illustrated herein implement a technical tool for testing and confirming content efficacy. This can be used to lower barriers to high-quality research in education by giving educators and school leaders a way to learn what works (and what does not) with little administrative overhead and a simple wizard-style set up. Thus, embodiments provide educators a simple platform to design and implement randomized controlled trials and A/B testing of learning material.

Embodiments offer educators a step-by-step guide to testing learning materials' efficacy in their classes. For students, embodiments are imperceptible as the students are randomly assigned to a group (e.g., A or B group, where A and B may be ‘control’ and ‘variation being tested’ respectively) and routed to the corresponding content in a learning management system (LMS). Learning materials remain seamlessly integrated via the LMS with the content efficacy testing.

At the end of each test of learning materials, educators view the results on a dashboard that helps them make sense of the findings. Educators can also download reports and datasets on demand for further analysis and reporting. Unlike other tools that are built into specific products or curriculum, embodiments use standardized interoperability tools to integrate with any standard compliant LMS. For example, some embodiments are configured to operate with LMSs that are compliant with Learning Tools Interoperability (LTI) 1.3 maintained by IMS Global Learning Consortium. Note that when examples of LTI are illustrated herein, it should be appreciated that in other embodiments, other communication protocols may be used in the alternative.

Embodiments are implemented to provide an evidence-based tech solution to alleviate the challenges educators and students face. Embodiments may implement a platform that enables multivariate testing and routing of LTI (or other learning standard communication protocol) based content to support continuous improvement. It acts as a proxy that resides between an LMS and LTI, or other content or content providers. When a student inside an LMS clicks an LTI-enabled (or similar) link, the student is invisibly routed through a random clinical trials (RCT) management system first. The link may be displayed in a user interface at the LMS, and may be a link to an educational module type. In some embodiments, the link may be a link that purports to be to a particular set of educational materials, even though a student selecting the link may result in the student being directed one randomly selected choice between two (or more) different sets of educational materials. The RCT management system receives an LTI launch event as a result of the student selecting the link, and then—based on the way that link was configured by an RCT management admin—redirects the student to one of several targets of downstream content (e.g., education modules for a course). This allows instructional designers, instructors, administrators, or anyone else interested in outcome-based learning to measure the effectiveness of content.

The RCT management system achieves this proxy behavior by receiving an initial LTI launch event from the LMS as a result of the student selecting the current link, looking up the student to see if they already have a profile with profile information for that link, using the configuration for the current link to determine which random group the student will be in, then replaying the LTI launch event into the selected downstream LTI resource (e.g., the randomly selected educational module).

With the RCT management system, users can conduct randomized clinical trials, A/B tests of two or more tools connected via an appropriate protocol, such as LTI, and any other multivariate analysis. Since the RCT management system also completes the LTI launch process by sending the resulting grade back up to the LMS, users can correlate gradebook outcomes with the different learning activities students were assigned.

While RCT management system configurations tend to be very developer-centric, embodiments illustrated herein expand the RCT management system by adding an elegant user interface, including a configuration interface and reporting dashboard interface, that simplifies the user experience for all audiences.

Embodiments may further be configured to interact with systems beyond those that are LTI compliant. For example, some embodiments are configured for integration with platforms incorporating an online suite of applications configured for blended learning comprising online learning combined with traditional place-based methods. One such platform is Google Classroom. Given Google Classroom's dispersion in K-12 environments, by integrating with such platforms, embodiments empower more learning tool research in that space.

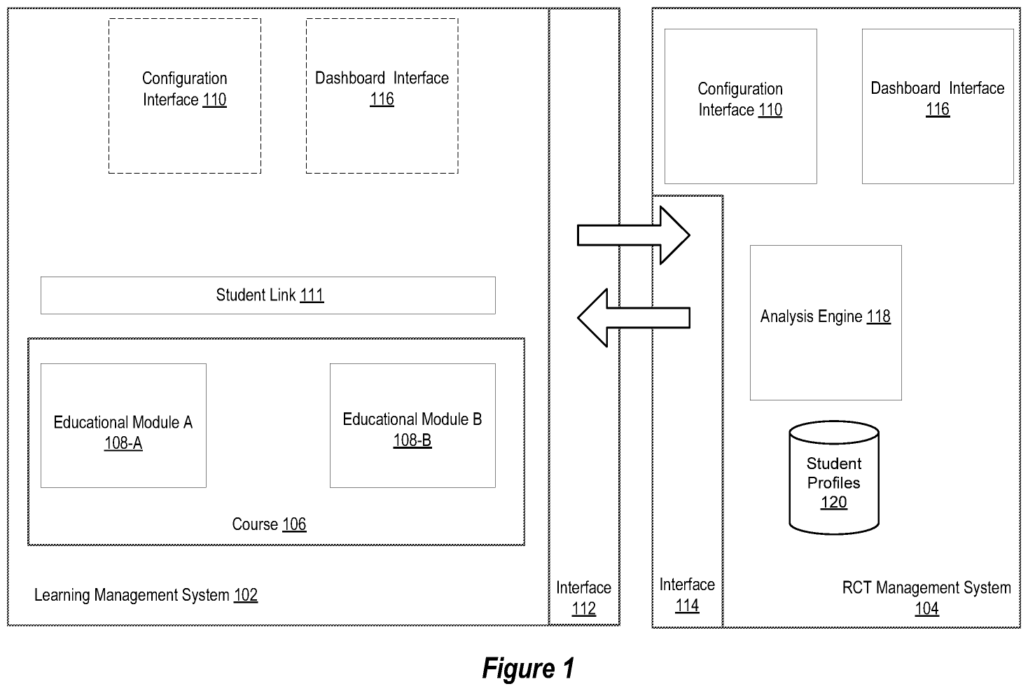

Referring now to FIG. 1, an example is illustrated. FIG. 1 illustrates a learning management system 102. The learning management system 102 is coupled to a random clinical trials (RCT) management system 104. These two systems can work in concert to both provide educational materials and allow for testing of variations in educational materials.

FIG. 1 illustrates that the learning management system 102 administers and potentially hosts various courses such as course 106. Note that while course 106 is shown as part of the learning management system 102, it should be appreciated that course 106 can be hosted on external systems and can be accessed using various interfaces. For example, in some embodiments, an interface that is compliant with LTI 1.3 may allow the learning management system 102 to access external databases having learning materials for the course 106.

In the example illustrated in FIG. 1, the course 106 is associated with two different educational modules, educational module A 108-A and educational module B 108-B. These educational modules represent control and variation educational modules respectively. In particular, students selected for a control group will be assigned to educational module A 108-A while students selected for a variation test group will be assigned to educational module B 108-B. Note that while only two educational modules are illustrated, it should be appreciated that in other embodiments multiple different educational modules may be implemented. For example, multiple variations can be tested at the same time.

Attention is now directed to the RCT management system 104. The RCT management system 104 includes a configuration interface 110. The configuration interface 110 is able to be accessed by users of the learning management system 102 (as illustrated by the dashed box shown in the learning management system 102). For example, an educator using the learning management system 102 can access the configuration interface 110 to enter parameters for a particular random clinical trial for course materials, such as the variations included in the educational module B 108-B.

As illustrated in FIG. 1, the learning management system 102 may be connected to the RCT management system 104 in a fashion that allows the configuration interface 110 to be displayed as if it were integral to the learning management system 102. In particular, the learning management system 102 includes an interface 112 that allows it to communicate with external systems. For example, the interface 112 may be an interface that is compliant with LTI 1.3. Alternatively, other interfaces may be used. Similarly, the RCT management system 104 also includes an interface 114. The interface 114 may be for example an interface that is compliant with LTI 1.3. This allows the RCT management system 104 to provide the configuration interface 110 to users at the learning management system 102.

In some embodiments, the configuration interface 110 allows a user, such as an educator, to select various educational modules for RCT testing, to define control versus variation modules, to select the desired sample size, to select evaluation criteria, etc. The information entered by the user in the configuration interface 110 is provided back to the RCT management system 104, and in particular to the analysis engine 118, which can then be used for implementing an RCT. The analysis engine 118 may be implemented using hardware and/or software as discussed below in the description of example computer systems.

Note that in some embodiments, the configuration interface 110 comprises a wizard style interface. In a wizard style interface, users are guided in a step-by-step fashion to input needed information for conducting an RCT. This ensures that lay users are properly guided to provide appropriate information to ensure that an appropriate RCT can be conducted. In this way, even the most novice user can design and implement a valid RCT.

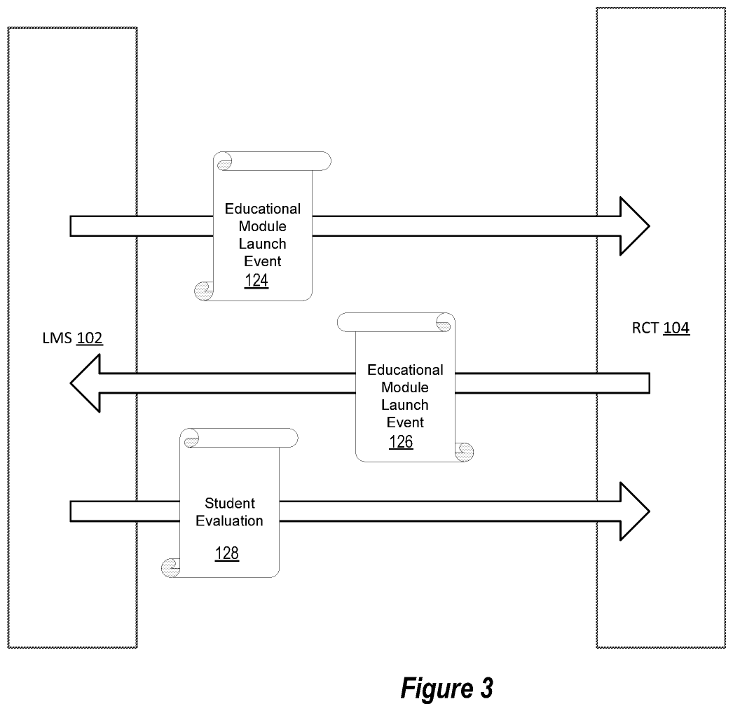

FIG. 1 illustrates that the learning management system 102 includes a student link 111. The student link 111 can be displayed in a user interface to students using the learning management system 102. Using this student link 111, the students can indicate a desire to participate in the course 106. While in an ordinary learning management system 102, the user would simply be directed to the appropriate educational module for the course, in embodiments herein, as discussed previously, communication, in the form of an educational module launch event 124 (see FIG. 3) is directed through the interface 112 to the interface 114 of the RCT management system 104 where the analysis engine 118 can perform various actions to assign a particular student to an appropriate educational module to randomly assign the student to either a control group of students assigned to the educational module A 108-A or to a variation group of students assigned to the educational module B 108-B (or other variation group as appropriate). The RCT management system 104 then replays an educational module launch event 126 back to the LMS 102 (as illustrated in FIG. 3). This is all transparent to the student as the student simply selects a link 111 for the course 106, administered by the learning management system 102, where the act of selecting the link 111, in isolation, has no effect on which particular module the student is ultimately directed.

Note, however, that the RCT management system includes a data store of student profiles 120. The student profiles 120 can include profile information about the students interacting with the LMS 102. For example, in some embodiments, the student profiles 120 can track previous student interactions so that intermittent interactions by students nonetheless have continuity in RCT testing. Thus, for example, if a student was previously assigned to a control group, and has therefore been previously accessing the educational module A 108-A, the student will continue to be directed to the educational module A 108-A, even when the student interacts with the LMS during different discrete sessions with system disconnects between sessions.

By the external RCT management system 104, and in particular the analysis engine 118, performing these assignments, appropriate standards for RCT testing can be maintained. In particular, double blind RCT testing can be performed where students can be assigned to conventional learning modules or to variation learning modules, the assignments can be random, investigator bias can be avoided inasmuch as masking procedures are maintained, and appropriate ethical bases for conduct of the research can be maintained inasmuch as the control is implemented at a remote analysis engine 118.

As students interact with the various educational modules (such as by entering notes into the LMS 102 (or associated module provider), providing indications of attentiveness by interaction or detection, completing assignments and projects on the LMS 102, taking quizzes and tests on the LMS 102, student evaluation data 128 (see FIG. 3) of the student with respect to course evaluation and completion learning objectives (based on the notes, attentiveness, assignments and projects, quizzes, tests, etc.) is provided through the interface 112 to the interface 114, and to the analysis engine 118. Here, the analysis engine can perform appropriate RCT categorization, analysis, or other activities.

FIG. 1 further illustrates that the RCT management system 104 includes a dashboard interface 116. The dashboard interface 116 is coupled to the analysis engine 118 to receive information from the analysis engine 118 when appropriate test data has been collected and organized. That is, the dashboard interface can display results to educators to help them make sense of findings of an RCT. As illustrated in FIG. 1, the dashboard interface 116 can be displayed within the learning management system 102. This can be done by exchanging information between the interfaces 112 and 114.

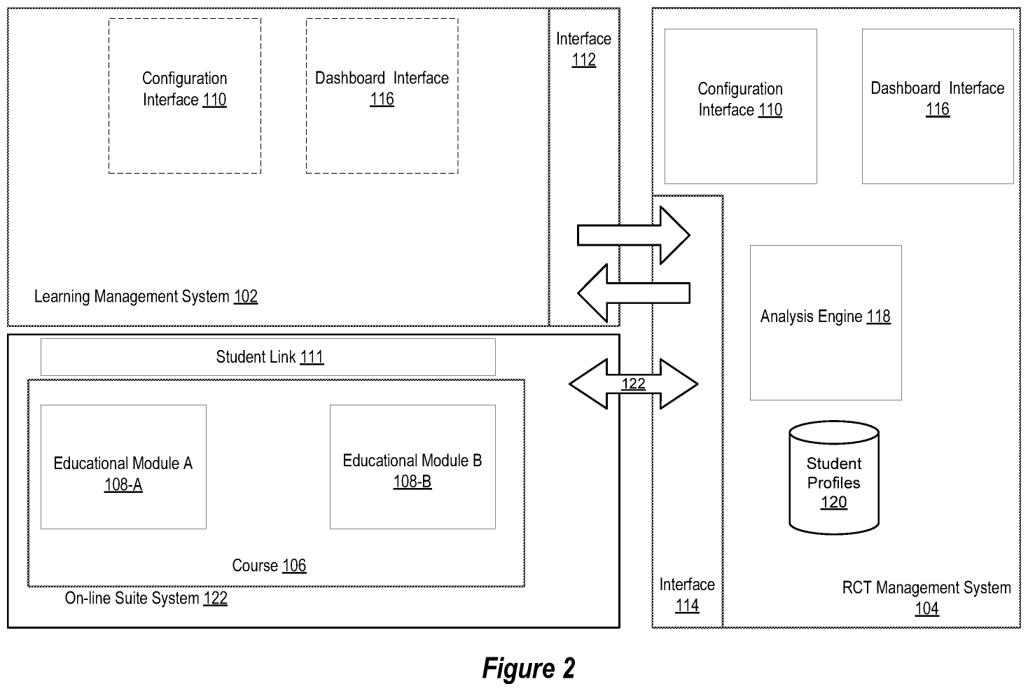

Note that in some embodiments, the RCT management system 104 is configured such that the communications interface 114 is configured to communicate with a platform incorporating an online suite of applications, illustrated as on-line suite system 122 in FIG. 2, configured for blended learning comprising online learning combined with traditional place-based methods. One such platform is Google Classroom. In these embodiments, the interface 114 is configured with appropriate authentication and launch flow as illustrated at 122. In the particular example of Google Classroom, embodiments, include a flow which includes launching an activity/assignment in Google Classroom into the RCT management system 104, entering the RCT management system 104 routing flow, launching the proper URL, and recording the course work and grade via the ‘studentSubmissions’ API. The RCT management system 104 can provide RCT results to the LMS 102 through the dashboard interface 116 using the interfaces 112 and 114.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

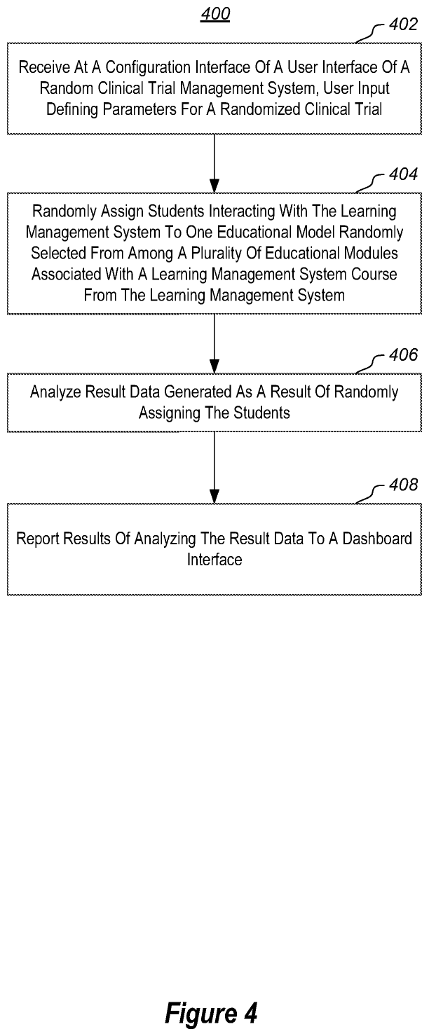

Referring now to FIG. 4, a method 400 is illustrated. The method 400 includes performing a random clinical trial in a learning management system environment. The method 400 includes receiving at a configuration interface of a user interface of a random clinical trial management system, user input defining parameters for a randomized clinical trial (act 402).

The method 400 further includes, at an analysis engine of the random clinical trial management system, based on user input received at the configuration interface, randomly assigning students interacting with the learning management system to one educational model randomly selected from among a plurality of educational modules associated with a learning management system course from the learning management system (act 404). The plurality of educational modules includes a control educational module, and at least one variation educational module correlated to the control educational module comprising a variation to be tested with respect to the control educational module.

The method 400 further includes, at the analysis engine of the random clinical trial management system, analyzing result data generated as a result of randomly assigning the students (act 406). Analyzing result data is performed based on user input received at the configuration interface.

The method 400 further includes, reporting results of analyzing the result data to a dashboard interface (act 408) The results of analyzing the result data is presented to a user at the learning management system. The results of analyzing the result data are correlated by educational modules comprising at least one correlation between the control educational module and at least one variation educational module. The results of analyzing the result data is provided through the dashboard interface via the communications interface to the user at the learning management system.

The method 400 may be practiced where receiving at the configuration interface of the user interface of the random clinical trial management system, user input defining parameters for the randomized clinical trial the configuration interface comprises receiving user input in a wizard interface.

The method 400 may be practiced where the communications interface is compliant with LTI 1.3.

The method 400 may further include the communications interface communicating with a platform incorporating an online suite of applications configured for blended learning comprising online learning combined with traditional place-based methods.

The method 400 may further include the communications interface receiving educational module launch events from the learning management system as a result of a student selecting a link for educational materials and the communications interface replaying an educational module launch event back to the learning management system to a selected educational module based on the results of randomly assigning students interacting with the learning management system to one of the education modules. In some such embodiments, the analysis engine looks up information about the student to identify profile information for the student correlated to the link, and wherein replaying the educational module launch event back to the learning management system to the selected educational module is based on the profile.

The method 400 may further include the communications interface receiving student evaluation information from the learning management system and the analysis engine generating the results of analyzing the result data based on the student evaluation information.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

Embodiments of the present invention may comprise or utilize a special purpose or general-purpose computer including computer hardware, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

The present invention may be embodied in other specific forms without departing from its characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A random clinical trial management system for implementing randomized clinical trials in educational systems, the system comprising:
a user interface, wherein the user interface comprises:
a configuration interface configured to receive user input defining parameters for a randomized clinical trial; and
a dashboard interface configured to output, to a user, results of the randomized clinical trial, wherein the results are correlated by educational modules comprising at least one correlation between a control educational module and at least one variation educational module;
the random clinical trial management system further comprising an analysis engine configured to:
analyze data from a plurality of educational modules associated with a learning management system course administered by a learning management system to generate the results of the randomized clinical trial, including the control educational module, and the at least one variation educational module correlated to the control educational module comprising a variation to be tested with respect to the control educational module, wherein analyzing is performed based on user input received at the configuration interface;
randomly assign students interacting with the learning management system to one of the educational modules in the plurality of educational modules based on user input received at the configuration interface; and
report the results of the randomized clinical trial to the dashboard interface, where the results of the randomized clinical trial can be presented to a user;
the random clinical trial management system further comprising a communications interface configured to be coupled to learning management systems, and configured to:
receive user input at the configuration interface from users of the learning management systems; and
provide the results of the randomized clinical trial through the dashboard interface to users at the learning management systems.

2. The random clinical trial management system of claim 1, wherein the configuration interface comprises a wizard interface.

3. The random clinical trial management system of claim 1, wherein the communications interface is compliant with LTI 1.3.

4. The random clinical trial management system of claim 1, wherein the communications interface is configured to communicate with a platform incorporating an online suite of applications configured for blended learning comprising online learning combined with traditional place-based methods.

5. The random clinical trial management system of claim 1, wherein:
the communications interface is configured to receive educational module launch events from the learning management system as a result of students selecting links for educational materials; and
the communications interface is configured to replay educational module launch events back to the learning management system to selected educational modules based on the results of randomly assigning students interacting with the learning management system to one of the education modules.

6. The random clinical trial management system of claim 5, wherein the analysis engine is configured to look up information about students interacting with the learning management system to identify profile information for students correlated to links, and wherein to replaying educational module launch events back to the learning management system to selected educational modules is based on the profiles.

7. The random clinical trial management system of claim 1, wherein:
the communications interface is configured to receive student evaluation information from the learning management system; and
the analysis engine is configured to generate the random clinical trial results based on the student evaluation information.

8. A method of performing a random clinical trial in a learning management system environment, the method comprising:
receiving at a configuration interface of a user interface of a random clinical trial management system, user input defining parameters for a randomized clinical trial;
at an analysis engine of the random clinical trial management system, based on user input received at the configuration interface, randomly assigning students interacting with the learning management system to one educational model randomly selected from among a plurality of educational modules associated with a learning management system course from the learning management system, including a control educational module, and at least one variation educational module correlated to the control educational module comprising a variation to be tested with respect to the control educational module;
at the analysis engine of the random clinical trial management system, analyzing result data generated as a result of randomly assigning the students, wherein analyzing result data is performed based on user input received at the configuration interface; and
reporting results of analyzing the result data to a dashboard interface, where the results of analyzing the result data is presented to a user at the learning management system, wherein the results of analyzing the result data are correlated by educational modules comprising at least one correlation between the control educational module and at least one variation educational module, wherein the results of analyzing the result data is provided through the dashboard interface via a communications interface to the user at the learning management system.

9. The method of claim 8, wherein receiving at the configuration interface of the user interface of the random clinical trial management system, user input defining parameters for the randomized clinical trial the configuration interface comprises receiving user input in a wizard interface.

10. The method of claim 8, wherein the communications interface is compliant with LTI 1.3.

11. The method of claim 8, further comprising the communications interface communicating with a platform incorporating an online suite of applications configured for blended learning comprising online learning combined with traditional place-based methods.

12. The method of claim 8, further comprising:
the communications interface receiving educational module launch events from the learning management system as a result of a student selecting a link for educational materials; and
the communications interface replaying an educational module launch event back to the learning management system to a selected educational module based on the results of randomly assigning students interacting with the learning management system to one of the education modules.

13. The method of claim 12, further comprising the analysis engine looking up information about the student to identify profile information for the student correlated to the link, and wherein replaying the educational module launch event back to the learning management system to the selected educational module is based on the profile.

14. The method of claim 8, further comprising:
the communications interface receiving student evaluation information from the learning management system; and
the analysis engine generating the results of analyzing the result data based on the student evaluation information.

15. A physical computer readable storage medium comprising computer executable instructions that when executed by one or more processors cause the one or more processors to perform the following:
receiving at a configuration interface of a user interface of a random clinical trial management system, user input defining parameters for a randomized clinical trial;
at an analysis engine of the random clinical trial management system, based on user input received at the configuration interface, randomly assigning students interacting with a learning management system to one educational model randomly selected from among a plurality of educational modules associated with a learning management system course from the learning management system, including a control educational module, and at least one variation educational module correlated to the control educational module comprising a variation to be tested with respect to the control educational module;
at the analysis engine of the random clinical trial management system, analyzing result data generated as a result of randomly assigning the students, wherein analyzing result data is performed based on user input received at the configuration interface; and
reporting results of analyzing the result data to a dashboard interface, where the results of analyzing the result data is presented to a user at the learning management system, wherein the results of analyzing the result data are correlated by educational modules comprising at least one correlation between the control educational module and at least one variation educational module, wherein the results of analyzing the result data is provided through the dashboard interface via a communications interface to the user at the learning management system.

16. The physical computer readable storage medium of claim 15, wherein receiving at the configuration interface of the user interface of the random clinical trial management system, user input defining parameters for the randomized clinical trial the configuration interface comprises receiving user input in a wizard interface.

17. The physical computer readable storage medium of claim 15, wherein the communications interface is compliant with LTI 1.3.

18. The physical computer readable storage medium of claim 15, further comprising computer executable instructions that when executed by the one or more processors cause the one or more processors to perform the communications interface communicating with a platform incorporating an online suite of applications configured for blended learning comprising online learning combined with traditional place-based methods.

19. The physical computer readable storage medium of claim 18, further comprising computer executable instructions that when executed by the one or more processors cause the one or more processors to perform:
the communications interface receiving educational module launch events from the learning management system as a result of a student selecting a link for educational materials; and
the communications interface replaying an educational module launch event back to the learning management system to a selected educational module based on the results of randomly assigning students interacting with the learning management system to one of the education modules.

20. The physical computer readable storage medium of claim 19, further comprising computer executable instructions that when executed by the one or more processors cause the one or more processors to perform the analysis engine looking up information about the student to identify profile information for the student correlated to the link, and wherein replaying the educational module launch event back to the learning management system to the selected educational module is based on the profile.
